(19) United States
(12) Patent Application Publication
(10) Pub. No.: US 2014/0249530 A1
(43) Pub. Date: Sep. 4, 2014

(54) CERCLAGE WIRE INSERTION DEVICE

(71) Applicants: George Babikian, Falmouth, ME (US); Nick Babikian, Palos Verdes Estates, CA (US); Raymond White, Portland, ME (US)

(72) Inventors: George Babikian, Falmouth, ME (US); Nick Babikian, Palos Verdes Estates, CA (US); Raymond White, Portland, ME (US)

(21) Appl. No.: 14/006,327

(22) PCT Filed: May 9, 2012
(86) PCT No.: PCT/US12/37004

§ 371 Date: May 21, 2014

Related U.S. Application Data
(60) Provisional application No. 61/499,493, filed on Jun. 21, 2011.
(60) Provisional application No. 61/484,893, filed on May 11, 2011.

(51) Int. Cl. A61B 17/82

(52) U.S. Cl. CPC A61B 17/82
USPC 606/74

(57) ABSTRACT

The invention is an insertion tool for wrapping an orthopedic wire around a bone. The tool has a memory loop made of memory-shaped material which, when unconstrained, curves to a pre-determined diameter. A push-pull mechanism in the tool body is used to selectively move the memory loop in and out of the tool body. A cutter is provided at the insertion end of the tool body, to cut through tissue close to the bone. When pushed out of the tool body in a direction tangential to the perimeter of a bone, the memory loop automatically curves and wraps around the bone. Orthopedic wire is then attached to the memory loop, which is then pulled back into the tool body, pulling the orthopedic wire around the bone. A cable adapter facilitates coupling any conventional orthopedic wire to the insertion tool.

### BACKGROUND INFORMATION

1. Field of the Invention

The invention relates to a device for wrapping an orthopedic wire around a bone.

2. Discussion of the Prior Art

It is a common procedure in orthopedic surgery to pass a cerclage wire or cable about a fractured bone, to bring the parts together so the bone will heal. One of the difficulties of passing a cerclage wire around a bone is that muscle tissue is attached directly to the bone. A so-called “wire passer” is used to punch through the muscle tissue and encircle the bone. The conventional wire passer device is typically a rigid hook with a fixed radius of curvature. The hook is pushed into muscle tissue surrounding the bone, so as to encircle the bone, the cerclage wire is attached to the end of the hook and the hook is then backed out, thereby pulling the cerclage wire around the bone. Because of the rigid diameter of the hook, a significant amount of tissue is damaged in the process. This damage impairs subsequent bone healing, because it is this tissue that is vital to keeping the bone alive and allowing healing.

Conventional multi-filament cerclage cables are smooth with a flat-topped cone head and thus are not fitted with a ready means of connecting to the cerclage insertion tool.

What is needed therefore is an insertion tool that minimizes damage to the tissue that surrounds the bone. What is further needed is a means of attaching cerclage wire to the insertion tool.

### BRIEF SUMMARY OF THE INVENTION

The invention is an insertion device for passing a cerclage wire about a bone. The insertion device is a wire passer or an insertion tool. A cable adapter may be used to attach the cerclage wire to the insertion tool. The insertion tool has a memory-shaped, curved memory loop that encircles the bone. The cerclage wire is coupled to the end of the memory loop, which is then retracted into the insertion tool, thereby pulling the cerclage wire around the bone. The cerclage wire may be attached to the cable adapter, rather than being coupled directly to the memory loop.

For ease of illustration, reference will be made hereinafter to a “proximal” end and a “distal” end of the various components. “Proximal” refers to the end of the tool or component that is closer to the operator of the tool and “distal” to the end that is farther from the operator when the insertion tool is used for its intended purpose.

The insertion tool has a push-pull means that is slideably held within a tool body. The memory loop is affixed to a distal end of the push-pull means. The push-pull means may be any suitable component that has the axial strength and rigidity to push the wire passer out of the tool body. Examples include, but are not limited to, a wire, a rod or tube, or a bar. A grasping means, such as a handle or knob, is attached to the proximal end of the push-pull means to allow the operator to manipulate the memory loop. The memory loop is a cold-forged metal, i.e., memory-shaped, such that, when unconstrained, it reverts to a shape that has a pre-determined radius of curvature that forms a circle of a certain diameter. Ideally, the radius of curvature corresponds approximately to the diameter of the bone being worked on. The proximal end of the memory-shaped wire threader or memory loop is affixed to the distal end of the push-pull means. As the push-pull means is pushed into the tool body, the length of the memory loop that is thereby exposed automatically curves to the desired diameter.

The distal end of the tool body has a cutting tool for cutting through muscle tissue that is attached to the bone. To use the insertion tool, an incision is made close to the bone. The distal end of the insertion tool with the cutting tool is inserted into the incision, with the longitudinal axis of the tool body held approximately tangential to the curved circumference of the bone. Pressure is exerted on the insertion tool, to push the cutting tool through the muscle at the bone. The handle is then pushed in, thereby forcing the push-pull means down toward the distal end of the tool body, to force the memory loop out of the tool body and around the bone. Once the loop has encircled the bone, the cerclage wire is attached to the end of the loop and the loop is then retracted back into the tool body, thereby pulling the cerclage wire around the bone.

The cable adapter according to the invention may be used to couple any conventional cerclage wire or orthopedic cable to the insertion tool. The cable adapter has an adaptable sleeve that is bonded at one end to a flexible lead. The sleeve is ideally a biaxial braided sleeve, i.e., having a structure that is commonly known as a “Chinese finger trap,” and is dimensioned to receive the end of an orthopedic cable. Applying tension in the axial direction of the sleeve causes the sleeve to tighten and firmly grasp the orthopedic cable.

The flexible lead is coupled to an insertion end of the memory loop that has been wrapped around the bone. A simple method of attaching the cable adapter to the loop is to use a malleable wire as the lead and fold the wire back and twist it about itself, similar to the way a “twist tie” is secured in place about a plastic bag or cables, etc. The lead has a smaller diameter than that of the orthopedic cable and securing the lead in this way to the wire passer provides an inexpensive yet reliable method of coupling the cable adapter to the wire passer that does not cause unnecessary damage to the tissue.

The cable adapter according to the invention is ideally a disposable item, although the scope of the invention does not exclude a re-usable cable adapter. As a single-use disposable item, it is desirable that the device be made of readily available materials and be inexpensive to manufacture.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is an exploded view of the insertion tool according to the invention.

FIG. 2 is a perspective view of the tool body.

FIG. 2A is a top plan view of the tool body.

FIG. 2B is a side plan view of the tool body.

FIG. 3 is a perspective view of the memory loop and the push-pull means.

FIG. 3A is a top plan view of FIG. 3.

FIG. 3B is side plan view of the push-pull means and memory loop.

FIG. 4 is a perspective view of the handle.

FIG. 4A is a rear plan view of the handle, showing the bores for attaching the handle to the push-pull means.

FIG. 5 is a perspective view of the insertion tool, with the handle pushed all the way into the tool body and the exposed memory loop curved.

FIG. 6 is a perspective view of the insertion tool, showing the handle pulled back and the wire passer retracted into the tool body.

FIG. 7 is a schematic view in cross-section of the insertion tool in an incision, showing the cutter passing through tissue and the memory loop curving around the bone.

FIG. 8 is a schematic illustration of the cable adapter according to the invention, showing the sleeve in its relaxed state.

FIG. 9 is a schematic illustration of the cable adapter of FIG. 8, showing an orthopedic cable being inserted into the cable adapter.

FIG. 10 is a schematic illustration of the cable adapter of FIG. 8, showing the cable adapter tightened about the orthopedic cable.

FIG. 11 is a partial cross-section of the extended wire passer, showing the cable adapter secured to the insertion end of the wire passer.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art. Furthermore, dimensions for the various components are indicated in the drawings. These dimensions are not intended to be limiting in any way, as the inventive concept for the tool according to the invention is not limited to any specific dimensions.

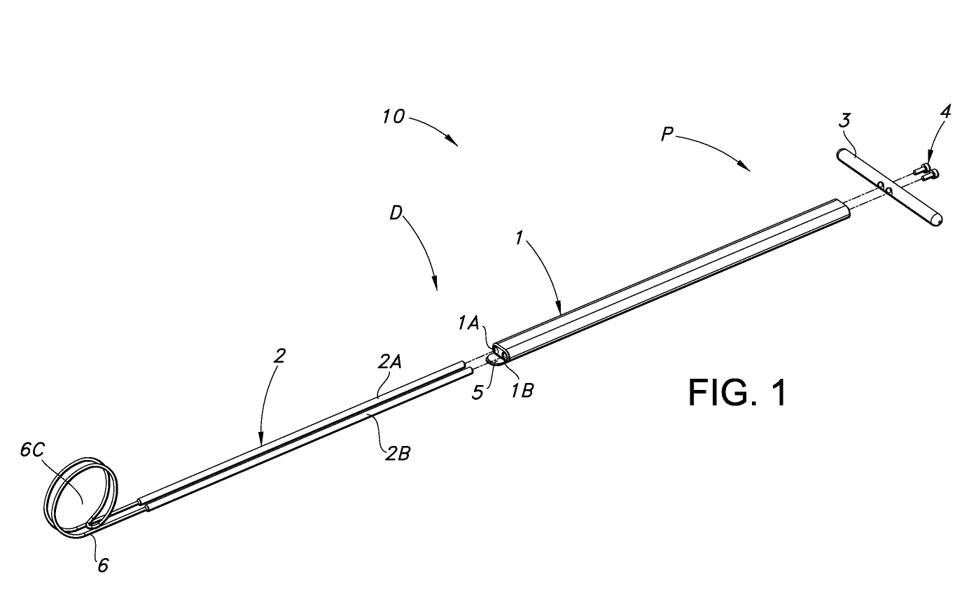

FIG. 1 is an exploded view of an insertion tool or wire passer 10 according to the invention. The tool 10 comprises a tool body 1, a push-pull means 2, and a memory loop or threader 6. For purposes of illustration, a proximal end P of the tool 10 is defined as the end that is closer to the operator when being inserted toward a bone B, and the distal end D as the end from which the wire passer 6 is extended.

The memory loop 6 is a memory-shaped wire that is cold-worked, so as to curve to a pre-defined radius of curvature when unconstrained, and flexible enough, so that it can be retracted into the tool body 1 by the push-pull means 2. In the embodiment shown, the push-pull means 2 is an axially rigid component, and specifically, includes two tubes or rods 2A and 2B. This component may be rigid or elastic in the lateral direction. The memory loop 6 is a dual-strand nitinol wire, the proximal ends of which are affixed to distal ends of the rods 2A and 2B. Although the push-pull means 2 is shown as having two separate insertion rods, it is understood that the push-pull means 2 may just as well be constructed as a single component, such as a bar, that is slideably held in the tool body 1, with the two ends of the memory loop 6 affixed to the distal end of the bar.

The proximal end of the push-pull means 2 is ideally fastened to a grasping means, such as a handle or knob 3 by means of a suitable fastener 4. Preferably, the fastener(s) 4 are removable fasteners, so that the insertion tool 10 may be taken apart for purposes of cleaning and sterilization. In this case, a mating threaded bore is provided at the proximal end of the rods 2A, 2B, to engage threaded fasteners in the handle 3. It is understood, however, that the insertion tool 10 may be constructed as a disposable tool, i.e., a single-use tool that is discarded. In this case, the fastening means may be a permanent connection, such as a heat-welded or fused connection of the push-pull means 2 to the handle 3, or the push-pull means and handle may be an integrated construction formed from a single mold or casting.

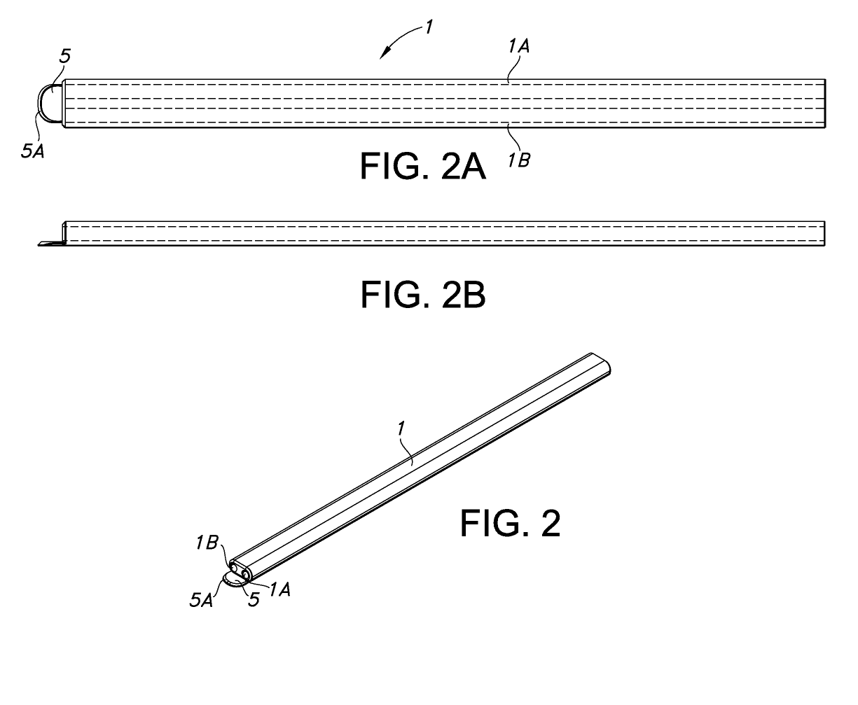

FIG. 2 is a perspective view of the tool body 1 and FIGS. 2A and 2B illustrate various details of the body 1. FIG. 2A is a top plan view of the tool body 1 and shows that, in this particular embodiment, the tool body 1 has two channels or through bores 1 A and 1B that are dimensioned to receive the two tubes 2A, 2B, respectively. The distal end of the tool body 1 has an insert blade or a cutting device 5, which is constructed as a scoop-like element with a cutting edge 5A for cutting through tissue that is attached to a bone B.

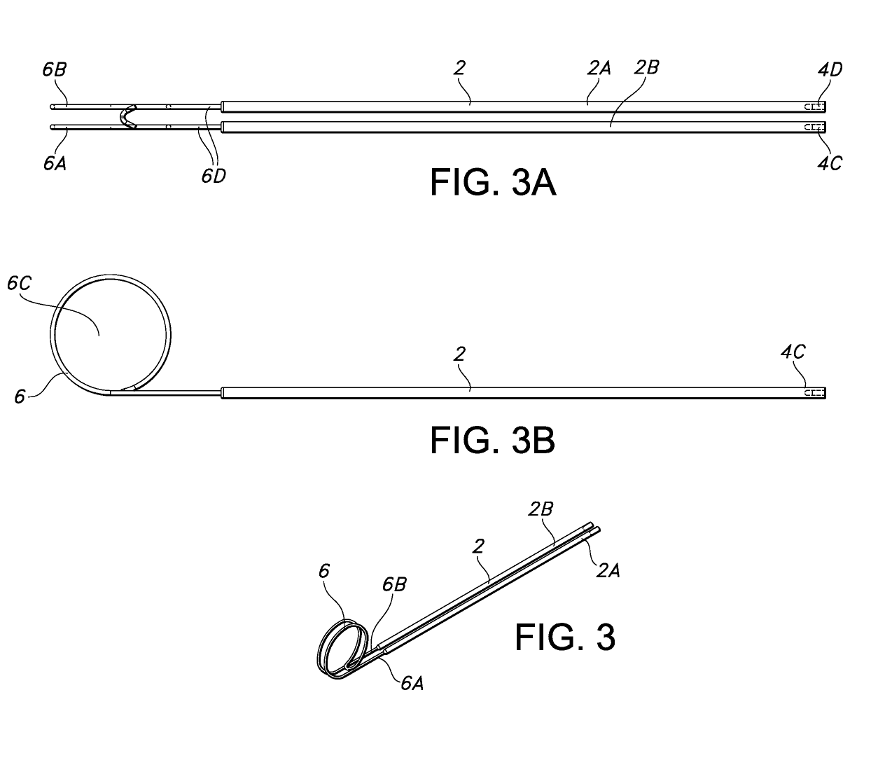

FIG. 3 is a perspective view of the memory loop 6 and push-pull means 2. In the embodiment shown, proximal ends of the wire threader 6A and 6B are inserted into the push-pull means 2A and 2B, respectively. The distal end of the wire passer 6 is memory-shaped to curve to a pre-defined radius of curvature 6C. FIG. 3A is a top plan view of the push-pull means 2 and the memory loop 6, showing the proximal end of the wire threader 6 extending from the distal end of the insertion tool 2, such that a distal portion of the memory loop 6 is unconstrained by the tool body 1 and free to curve to the pre-defined radius of curvature 6C. The ends of the wire 6D at the proximal end of the wire threader 6 are affixed in the push-pull means 2 by some means, such as an adhesive means or other mechanical means, for example, a set screw, a snap fastener, or a friction-fit. A series of wire threaders or loops 6, each curving to a different radius of curvature, may be made available for use with a single insertion tool 10. In this case, a surgeon would select a memory loop 6 having a radius of curvature that corresponds essentially to the diameter of the bone B to be encircled with the orthopedic wire.

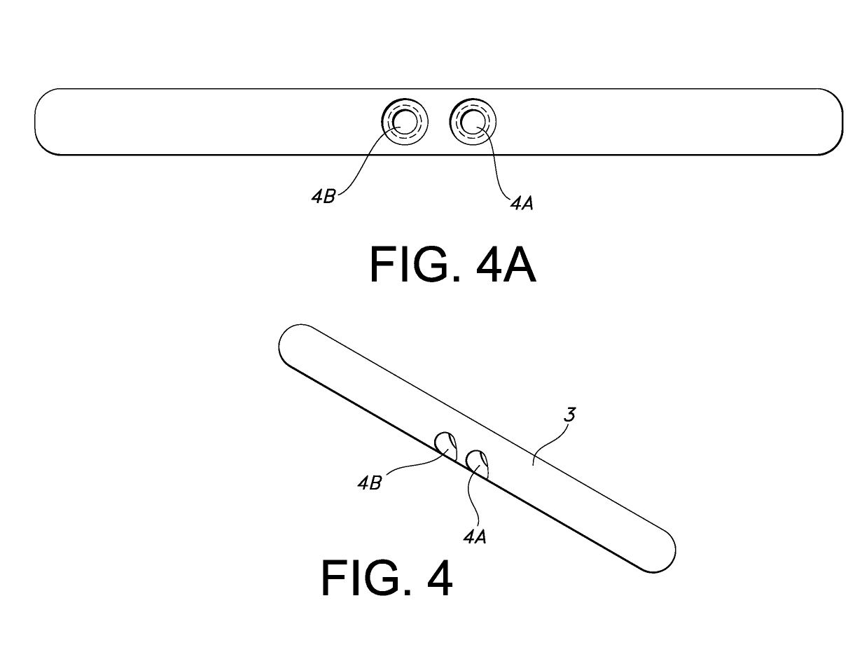

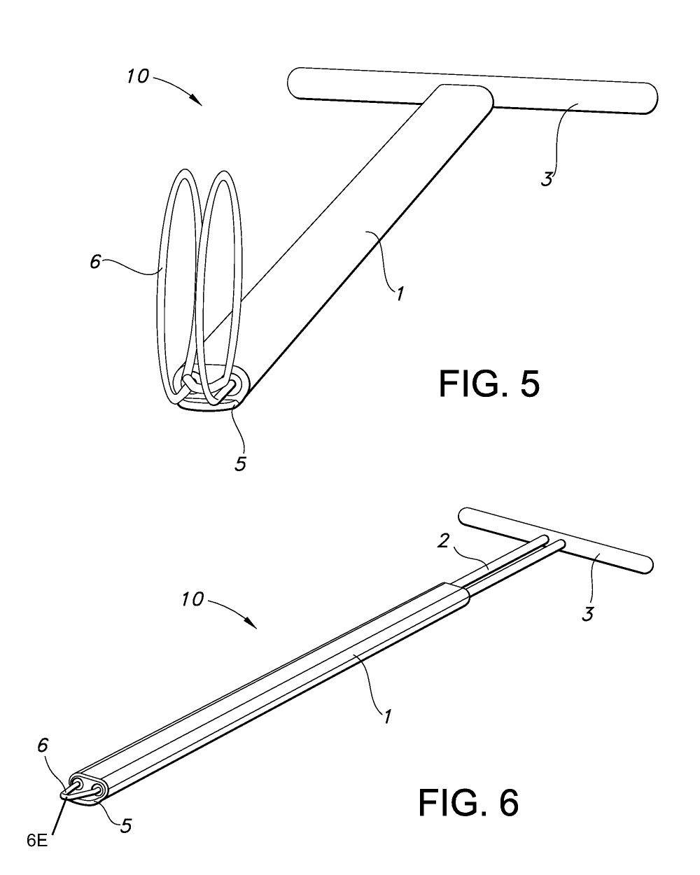

FIG. 4 is a perspective view of the handle 3. FIG. 4A shows two threaded bores 4A, 4B, for receiving a threaded fastener 4, shown in FIGS. 1 and 5. In the embodiment shown, the push-pull means 2 is detachably attached to the handle 3. The proximal ends of the push-pull means 2 have threaded bores 4C and 4D for receiving threaded fastening means that are attached to the handle 3.

FIGS. 5 and 6 illustrate two different positions of the insertion tool 10. FIG. 5 shows the handle 3 pushed in to its limit within the tool body 2. The handle 3 forces the push-pull means 2 to slide toward the distal end of the tool body 1, thereby pushing the wire threader 6 out of the insertion tool 10. As shown, the memory loop 6 curves to a particular radius of curvature. FIG. 6 shows the handle 3 pulled out of the proximal end of the tool body 2, thereby retracting the memory loop 6 back into the tool body 2.

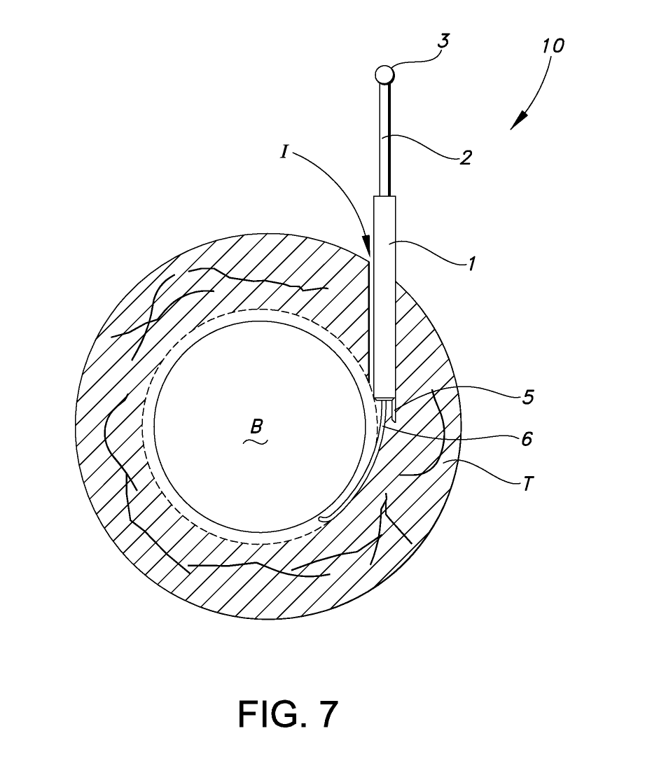

FIG. 7 is a schematic illustration of the insertion tool 10 inserted into an incision I. The incision I is made close to the bone B, such that when the tool 10 is inserted, the tool body 2 is held so that a longitudinal axis of the tool body 2 is approximately tangential to the circumference of the bone B. Tissue T, including muscle, is attached to the bone B. In this schematic illustration, the bone and the surrounding tissue are drawn as round elements. It is understood, however, that the contour of the bone B may be very different from this illustration. The cutting device 5 is shown passing through muscle tissue that is attached to the bone. The cutter 5 is a fixed component of the tool body 1 and does not travel around the bone with the memory loop 6, but is used only to cut through muscle tissue. After the initial insertion, the operator of the insertion tool 10 begins pushing the handle 3 into the tool body 1. This forces the memory loop 6 out of the tool body 2. Because the radius of curvature of the memory loop 6 has been selected to closely correspond to the size of the bone, it will wrap closely around the bone B, as the handle 3 is pushed farther into the tool body 2. The dashed lines indicate approximately the path the wire passer 6 will follow.

Once the memory loop 6 has encircled the bone B, a cerclage wire is coupled to a coupling end 6E of the passer 6. The handle 3 is then retracted, thereby pulling the memory loop 6 back into the tool body 2 and simultaneously pulling the cerclage wire around the bone. Various methods may be used to attach the cerclage wire to the memory loop 6. For example, one end of a suture may be tied to the coupling end 6E of the memory loop 6 and the cerclage wire coupled or tied to the other end of the suture, so that is pulled around the bone as the memory loop 6 is retracted. The cerclage wire may also be coupled directly with the memory loop 6. For example, the cerclage wire may have a contoured coupler at the end of the wire that is couplable with the threader 6, such that it is securely held by the threader when the threader is pulled back into the tool body. For example, a front face of the contoured coupler may be rounded, so that it snaps easily between the dual-strand threader 6 at the insertion or coupling tip 6E and is captured there, and the rear face may have an angled or catch contour, that prevents the coupler from inadvertently slipping out of the coupling tip 6E. Another example of a coupler is a ball that is small enough to insert through the two wire strands above the coupling tip 6E, yet is securely held in place between the two strands at the coupling tip.

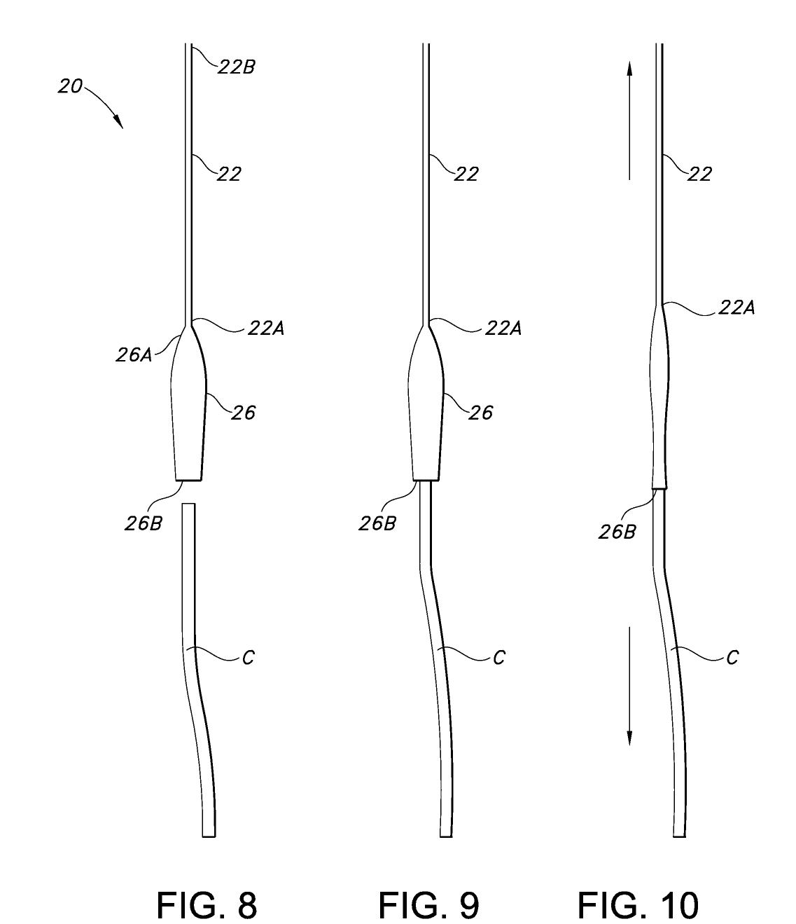

The process of attaching the orthopedic cable C to the memory loop 6 is simplified with use of the cable adapter 20 according to the invention, shown in FIGS. 8-10. The cable adapter 20 comprises a lead 22 to which an adaptable sleeve 26 is affixed. A first end 16A of the sleeve 26 is affixed to a first end 22A of the lead 22. The sleeve 26 may be permanently affixed, for example, by means of a bonding agent, adhesive, or other means, or removably affixed.

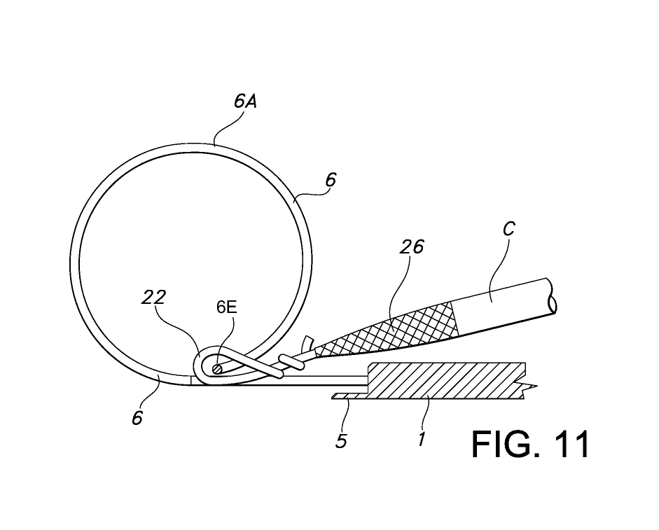

FIG. 11 illustrates the use of the cable adapter 20 with the insertion tool 10. The cable adapter 20 and the insertion tool 10 are used together to pull an orthopedic cable C around a bone. After the memory loop 6 has encircled the bone, i.e., when the loop is fully extended from the tool body 2, the lead 22 is secured to the insertion tip 6E of the memory loop 6. FIG. 11 is a partial cross-sectional view of the looped memory loop 6, with the cut at the coupling end 6E of the memory loop 6. In the embodiment shown, the lead 22 is a malleable wire that is bent around the tip 6E and its free end folded and pressed against or twisted about the itself, to secure the cable adapter 20 to the insertion tool 10. As the memory loop 6 is retracted into the tool body 3, the cable adapter 20 is pulled back around the bone, pulling the orthopedic cable C with it.

The adaptable sleeve 26 may be constructed of any suitable material that provides the desired adaptability in diameter so that it holds the cable C, such as, for example, a cylindrical, helically wound braided material, such as biaxial braided tubing. A material that is suitable for surgical purposes is a commercially available braided nylon, for example, Cortland Slip On Leader Loop connector braiding. Other biaxial braided sleeves may be used, of course, but this Leader Loop has shown that only a relatively short length of braid is required. For example, an overall length of two inches, with one inch covering the wire, was proved sufficient to provide a secure hold on the wire. The braided sleeve 26 has a certain diameter in its relaxed or resting state, that is, when no tensile or compressive forces are applied in the axial direction of the sleeve. Inherent to the braided structure is an elasticity that renders the sleeve well suited for use as a cable adapter. When tensile forces are exerted in the axial direction of the sleeve, its length increases and diameter decreases. Conversely, when compressive forces are applied in the axial direction, the length decreases and the diameter increases. As a result, the braided sleeve 26 is able to accommodate cables of various diameters, and, when a tensile force is exerted on the insertion tool 10, the tension is passed on to the sleeve, which then tightens about the orthopedic cable C. The force of friction exerted by the sleeve 26 on the cable C has sufficient holding strength to securely hold the cable while it is being pulled around the bone. Ideally, the sleeve 26 has a diameter in its relaxed state that is great enough to encompass all conventional orthopedic cables. It is within the scope of the invention, however, to provide the cable adapter in various sizes, to better accommodate cables with very small or very large diameters.

Specific materials for the insertion tool 10 have not been defined, with the exception of the memory loop 6, because any material that is suitable for the particular function or component may be used. The scope of the invention is, however, not necessarily limited to the use of nitinol for the memory loop 6. At the time the invention was conceived, nitinol was the only metal with the necessary elastic properties to glide around the bone with a specific curvature. Should other materials become available that have the desired properties for the memory loop, it understood that it is within the scope of the invention to use such materials. It is desirable that the tool 10 be rigid and that there be little wobble on the memory loop 6 as it is forced around the bone, in order to keep the travel path of the loop 6 as narrow as possible, to keep tissue damage to a minimum. For this reason, the embodiment shown in the figures is made of stainless steel and the tolerances between the push-pull means and the tool body are kept as close as economically feasible. Also, the tool body 1 is rectangular in shape, rather than round, which provides a cue to the surgeon to maintain the body 1 in a particular orientation to reduce wobble. Other suitable materials, at least for the cutter and tool body, include titanium.

It is understood that the embodiments described herein are merely illustrative of the present invention. Variations in the construction of the insertion tool and cable adapter may be contemplated by one skilled in the art without limiting the intended scope of the invention herein disclosed and as defined by the following claims.

What is claimed is:

1. An insertion tool for wrapping an orthopedic cable around a bone, the insertion tool comprising:
a tool body having an insertion end;
push-pull means that is slideably assembled in the tool body; and
a memory loop that is affixed to the push-pull means;
wherein the memory loop is a memory-shaped, dual-strand wire that curves to a pre-defined radius of curvature when unconstrained, the dual-strand wire having two wire strands and a coupling tip that connects a distal end of the two wire strands, a proximal end of the two wire strands being affixable to the push-pull means; and
wherein the memory loop is selectively manipulable by the push-pull means to extend from an insertion end of the tool body and wrap around the bone and to retract back into the tool body.

2. The insertion tool of claim 1, wherein the push-pull means includes a pair of push elements that are axially rigid and wherein the memory loop is affixed to the push elements.

3. The insertion tool of claim 2, wherein the tool body includes two channels, each channel dimensioned to receive a corresponding one of the push elements.

4. The insertion tool of claim 1, wherein the memory loop is removably affixed to the push-pull means.

5. The insertion tool of claim 1, wherein the memory loop is permanently affixed to the push-pull means.

6. The insertion tool of claim 1, further comprising a manipulation means that is affixed to the push-pull means, for slideably manipulating the push-pull means in the tool body.

7. The insertion tool of claim 6, wherein the manipulation means is a T-shaped handle that is affixed to the push-pull means.

8. The insertion tool of claim 6, wherein the manipulation means is a knob.

9. The insertion tool of claim 1, further comprising a cutting device provided at the insertion end of the tool body for cutting through tissue.

10. The insertion tool of claim 1, further comprising a cable adapter for attaching an orthopedic wire to the coupling tip of the wire passer, the cable adapter having an adaptable sleeve for entraining the orthopedic cable and an attachment means for removably affixing the adaptable sleeve to the memory loop.

11. The insertion tool of claim 10, wherein the adaptable sleeve is a biaxially braided sleeve having a diameter that reduces in size when a tensile force is applied to the adaptable sleeve.

12. The insertion tool of claim 10, wherein the attachment means is a flexible lead.

13. The insertion tool of claim 12, wherein the flexible lead is a malleable wire.

14. The insertion tool of claim 12, wherein the flexible lead is a tie.

15. The insertion tool of claim 10, wherein the attachment means is a clip.

16. The insertion tool of claim 10, wherein the attachment means is a ball that is catchable in the coupling tip.

17. A cable adapter for use with an insertion tool for wrapping an orthopedic wire around a bone, the cable adapter having an adaptable sleeve for entraining the orthopedic wire and an attachment means for removably affixing the adaptable sleeve to the insertion tool.

18. The cable adapter of claim 17, wherein the adaptable sleeve is a biaxially braided sleeve having a diameter that reduces in size when a tensile force is applied to the adaptable sleeve.

19. The cable adapter of claim 17, wherein the attachment means is a flexible lead.

20. The cable adapter of claim 17, wherein the attachment means is a clip.
